(19) United States
(12) Patent Application Publication
(10) Pub. No.: US 2008/0125452 A1
(43) Pub. Date: May 29, 2008

(54) Indolopyridines,Benzofuranopyridines and Benzothienopyridines

(75) Inventors: Matthias Vennemann, Kontanz (DE); Thomas Bar, Reichenau (DE); Jurgen Braunger, Konstanz (DE); Petra Gimmnich, Konstanz (DE); Thomas Beckers, Konstanz (DE); Mathias Schmidt, Konstanz (DE); Thomas Ciossek, Ravensburg (DE); Sandra Nappe, Konstanz (DE)

Correspondence Address: NATH & ASSOCIATES PLLC, 112 South West Street, Alexandria, VA, 22314, US

(73) Assignee: ALTANA PHARMA AG, Konstanz (DE)

(21) Appl. No.: 11/795,762

(22) PCT Filed: Jan. 26, 2006
(86) PCT No.: PCT/EP06/50465

§ 371 Date: Aug. 31, 2007

(30) Foreign Application Priority Data
Jan. 28, 2005 (EP) 05100594.0
Feb. 9, 2005 (EP) 05100913.2

(51) Int. Cl. A61K 31/4745; C07D 491/14; C07D 498/14; C07D 487/14

(52) U.S. Cl. 514/287; 546/64

(57) ABSTRACT

Compounds of a certain formula (I),

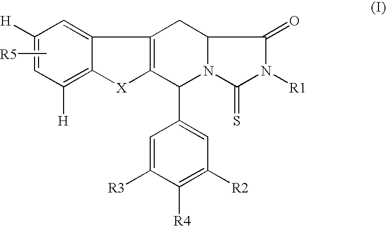

in which R1, R2, R3, R4, R5 and X have the meanings indicated in the description, are novel effective compounds with anti-proliferative and/or apoptosis inducing activity.

### FIELD OF APPLICATION OF THE INVENTION

The invention relates to indolopyridines, benzofuranopyridines and benzothienopyridines, which display cell-cycle dependent, anti-proliferative and apoptosis inducing activity, and which can be used in the pharmaceutical industry for the production of pharmaceutical compositions.

The invention also relates to the use of these derivatives for the therapy of hyperproliferative diseases, in particular human cancer.

### KNOWN TECHNICAL BACKGROUND

Cancer chemotherapy was established with the alkylating agent Cyclophosphamide (Endoxan®), an oxazaphosphorin pro-drug activated preferentially in the tumor. The target of alkylating agents like Cyclophosphamide is DNA and the concept, that cancer cells with uncontrolled proliferation and a high mitotic index are killed preferentially, proved to be very successful. Standard cancer chemotherapeutic drugs finally kill cancer cells upon induction of programmed cell death (“apoptosis”) by targeting basic cellular processes and molecules. These basic cellular processes and molecules include RNA/DNA (alkylating and carbamylating agents, plain analogs and topoisomerase inhibitors), metabolism (drugs of this class are named anti-metabolites and examples are folic acid, purin and pyrimidine antagonists) as well as the mitotic spindle apparatus with αβ-tubulin heterodimers as the essential component (drugs are categorized into stabilizing and destabilizing tubulin inhibitors; examples are Taxol/Paclitaxel®, Docetaxel/Taxotere® and vinca alkaloids).

A subgroup of proapoptotic anticancer agents target cells preferentially in mitosis. In general these agents do not induce apoptosis in non-dividing cells, arrested in the G0, G1 or G2 phase of the cell division cycle. In contrast, dividing cells going through mitosis (M-phase of the cell division cycle), are killed efficiently by induction of apoptosis by this subgroup agents. Therefore, this subgroup or class of anti-cancer agents is described as cell-cycle specific or cell-cycle dependent. Tubulin inhibitors, with Taxol (Paclitaxel®) as a prominent example, belong to this class of cell-cycle specific, apoptosis inducing anti-cancer agents.

EP357122 contains, inter alia, indolopyridine, benzofuranopyridine and benzothienopyridine derivatives as cytostatic compounds.

In the International Applications WO9632003 and WO0228865 indolopyridine derivatives are described with PDE inhibitory activity.

In the document Hotha et al., Angew. Chem. 2003, 115, 2481-2484 the indolopyridine compound HR22C16 is described as inhibitor of cell division by targeting Eg5.

In the US-application US 2005/0004156 indolopyridine derivatives, specifically monastroline derivatives, are described as Eg5 inhibitors.

In Bioorg. Med. Chem. 13 (2005) 6094-6111 tetrahydro-β-carbolines are described as Eg5 inhibitors. In the International Application WO 2004/004652, inter alia, trans-10-(3-hydroxy-phenyl)-2-methyl-3a,4,9,10-tetrahydro-2,9,10a-triaza-cyclopenta[b]fluorene-1,3-dione is described in a crystallized complex with the kinesin spindle protein (KSP).

In J. Org. Chem., vol. 59, no. 6,1994, p. 1583-1585 and Chem. Pharm. Bull., vol. 42, no. 10, 1994, p. 2108-2112 the reaction of tetrahydro-β-carboline-3-carboxylic acids with isocyanates and isothiocyanates is described.

In J. Med. Chem., vol. 46, no. 21, 2003, p. 4525-4532 indolopyridine derivatives are described with PDE5 inhibitory activity.

The International Application WO 2005/089752 describes tetracyclic carboline derivatives as inhibitors of VEGF production.

### DESCRIPTION OF THE INVENTION

It has now been found that the purposively selected indolopyridine, benzofuranopyridine and benzothienopyridine derivatives, which are described in greater details below, differ profoundly from previously individualized compounds and have surprising and particularly advantageous properties. Thus, for example, the compounds according to this invention are potent and highly efficacious inhibitors of cellular (hyper)proliferation and/or cell-cycle specific inducers of apoptosis in cancer cells. Therefore, unanticipatedly, these compounds can be useful for treating (hyper)proliferative diseases and/or disorders responsive to the induction of apoptosis, in particular cancer. By having a cell-cycle specific mode of action, these derivates should have a higher therapeutic index compared to standard chemotherapeutic drugs targeting basic cellular processes like DNA replication or interfering with basic cellular molecules like DNA.

Thus, for example, the compounds according to this invention are expected to be useful in targeted cancer therapy.

The invention thus relates to compounds of formula I

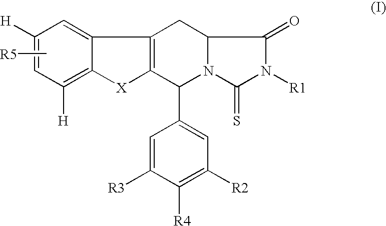

in which

### R1 is methyl, ethyl, isopropyl or cyclopropyl,

### R2 is methyl, ethyl, halogen, trifluoromethyl, ethoxy or methoxy,

### R3 is methyl, ethyl, halogen, trifluoromethyl, ethoxy or methoxy,

### R4 is hydrogen,

### X is oxygen (O), sulphur (S), or NH,

### R5 is hydrogen, methyl, ethyl, halogen, trifluoromethyl, ethoxy or methoxy,

and the salts, stereoisomers and the salts of the stereoisomers of these compounds.

Halogen within the meaning of the present invention is iodine or, in particular, bromine, or, in more particular, chlorine or fluorine.

Suitable salts for compounds according to this invention—depending on substitution—are all acid addition salts or all salts with bases. Particular mention may be made of the pharmacologically tolerable inorganic and organic acids and bases customarily used in pharmacy. Those suitable are, on the one hand, water-insoluble and, particularly, water-soluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic add, oxalic acid, tartaric acid, embonic acid, stearic acid, toluenesulphonic acid, methanesulphonic acid or 3-hydroxy-2-naphthoic acid, the acids being employed in salt preparation—depending on whether a mono- or polybasic acid is concerned and depending on which salt is desired—in an equimolar quantitative ratio or one differing therefrom.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

Pharmacologically intolerable salts, which can be obtained, for example, as process products during the preparation of the compounds according to this invention on an industrial scale, are converted into pharmacologically tolerable salts by processes known to the person skilled in the art.

According to expert's knowledge the compounds of formula I according to this invention as well as their salts may contain, e.g. when isolated in crystalline form, varying amounts of solvents. Included within the scope of the invention are therefore all solvates and in particular all hydrates of the compounds of formula I according to this invention as well as all solvates and in particular all hydrates of the salts of the compounds of formula I according to this invention.

The substituent R5 of compounds of formula I can be attached in the 5- or 7-position of the scaffold. In one embodiment, R5 is different from hydrogen and is attached in the 5-position, in another embodiment, R5 is different from hydrogen and is attached in the 7-position of the scaffold, and in yet another embodiment R5 is hydrogen.

The compounds of formula I are chiral compounds having chiral centers in positions 3a and 10.
Numbering:

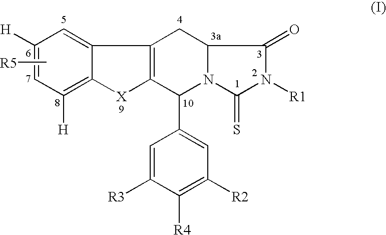

The invention includes all conceivable stereoisomers, like e.g. diastereomers and enantiomers, in substantially pure form as well as in any mixing ratio, including the racemates, as well as the salts thereof.

Preference is given hereby to compounds of formula I, which have with respect to the positions 3a and 10 the same configuration as shown in formula I*.

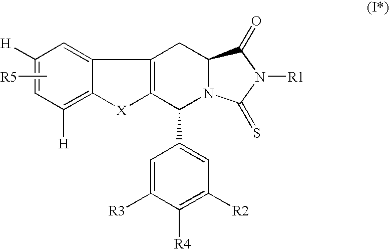

In compounds of formula I* the configuration—according to the rules of Cahn, Ingold and Prelog—is S in the 3a position and R in the 10 position.

Further on, compounds of the formula I also to be mentioned are those which have, with respect to the positions 3a and 10, the same configuration as shown in formula I**, I*** or I****:

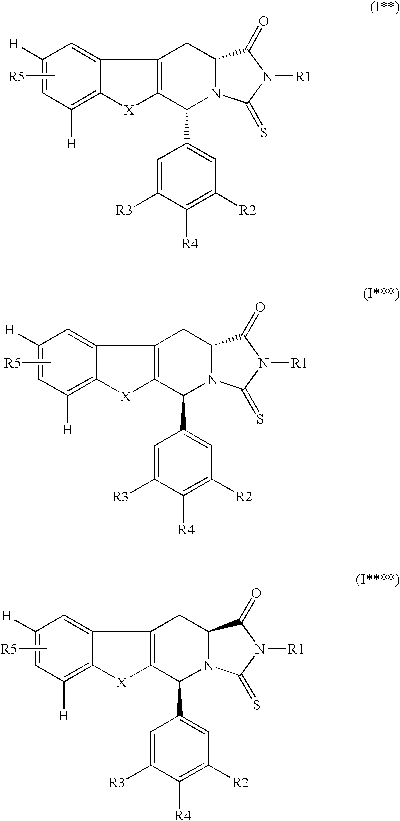

In compounds of formula I** the configuration—according to the rules of Cahn, Ingold and Prelog—is R in the 3a position and R in the 10 position.

In compounds of formula I*** the configuration—according to the rules of Cahn, Ingold and Prelog—is R in the 3a position and S in the 10 position.

In compounds of formula I**** the configuration—according to the rules of Cahn, Ingold and Prelog—is S in the 3a position and S in the 10 position.

In general, enantiomerically pure compounds of this invention can be prepared according to art-known processes, such as e.g. via asymmetric syntheses, for example by preparation and separation of appropriate diastereoisomeric compounds or by using chiral synthons or chiral reagents; by chromatographic separation on chiral separating columns; by means of salt formation of the racemic compounds with optically active acids or bases, subsequent resolution of the salts and release of the desired compound from the salt; by derivatization with chiral auxiliary reagents, subsequent diastereomer separation and removal of the chiral auxiliary group; or by (fractional) crystallization from a suitable solvent.

Preferably, enantiomerically pure compounds can be obtained starting from known enantiomerically pure starting compounds via synthesis of diastereomeric intermediates which can be separated by known methods (e.g. by chromatographic separation or crystallization), or by chromatographic resolution of the corresponding racemate on an appropriate chiral separating column.

The enantiomers having the formula I* and the salts thereof are a preferred part of the invention.

In the context of this invention, hyperproliferation and analogous terms are used to describe aberrant/dysregulated cellular growth, a hallmark of diseases like cancer. This hyperproliferation might be caused by single or multiple cellular/molecular alterations in respective cells and can be, in context of a whole organism, of benign or malignant behaviour. Inhibition of cell proliferation and analogous terms is used herein to denote an ability of the compound to retard the growth of and/or kill a cell contacted with that compound as compared to cells not contacted with that compound. Most preferable this inhibition of cell proliferation is 100%, meaning that proliferation of all cells is stopped and/or cells undergo programmed cell death. In some preferred embodiments the contacted cell is a neoplastic cell. A neoplastic cell is defined as a cell with aberrant cell proliferation. A benign neoplasia is described by hyperproliferation of cells, incapable of forming an aggressive, metastasizing tumor in-vivo. In contrast, a malignant neoplasia is described by cells with different cellular and biochemical abnormalities, e.g. capable of forming tumor metastasis. The acquired functional abnormalities of malignant neoplastic cells (also defined as “hallmarks of cancer”) are replicative potential (“hyperproliferation”), self-sufficiency in growth signals, insensitivity to anti-growth signals, evasion from apoptosis, sustained angiogenesis and tissue invasion and metastasis.

Inducer of apoptosis and analogous terms are used herein to identify a compound which executes programmed cell death in cells contacted with that compound. Apoptosis is defined by complex biochemical events within the contacted cell, such as the activation of cystein specific proteinases (“caspases”) and the fragmentation of chromatin. Induction of apoptosis in cells contacted with the compound might not necessarily be coupled with inhibition of cell proliferation. Preferably, the inhibition of cell proliferation and/or induction of apoptosis is specific to cells with aberrant cell growth (hyperproliferation). Thus, compared to cells with aberrant cell growth, normal proliferating or arrested cells are less sensitive or even insensitive to the proliferation inhibiting or apoptosis inducing activity of the compound. Finally, cytotoxic is used in a more general sense to identify compounds which kill cells by various mechanisms, including the induction of apoptosis/programmed cell death in a cell cycle dependent or cell-cycle independent manner.

Cell cycle specific and analogous terms are used herein to identify a compound as inducing apoptosis only in continuously proliferating cells actively passing a specific phase of the cell cycle, but not in resting, non-dividing cells. Continuously proliferating cells are typical for diseases like cancer and characterized by cells in all phases of the cell division cycle, namely in the G (“gap”) 1, S (“DNA synthesis”), G2 and M (“mitosis”) phase.

Compounds according to this invention more worthy to be mentioned are those compounds of formula I*,

in which

### R1 is methyl or ethyl,

### R2 is methyl, chlorine, fluorine, trifluoromethyl or methoxy,

### R3 is methyl, chlorine, fluorine, trifluoromethyl or methoxy,

### R4 is hydrogen,

### X is oxygen, sulphur or NH,

### R5 is hydrogen, methyl, chlorine, fluorine or trifluoromethyl,

and the salts of these compounds.

Compounds according to this invention further more worthy to be mentioned are those compounds of formula I*,

in which

### R1 is methyl,

### R2 is methyl, chlorine, fluorine, trifluoromethyl or methoxy,

### R3 is methyl, chlorine, fluorine, trifluoromethyl or methoxy,

### R4 is hydrogen,

### X is oxygen, sulphur or NH,

### R5 is hydrogen, methyl or fluorine,

and the salts of these compounds.

Compounds according to this invention in particular worthy to be mentioned are those compounds of formula I*,

in which

### R1 is methyl,

### R2 is methyl, chlorine, fluorine or methoxy,

### R3 is methyl, chlorine, fluorine or methoxy,

### R4 is hydrogen,

### X is oxygen, sulphur or NH,

### R5 is hydrogen, methyl or fluorine,

and the salts of these compounds.

Compounds according to this invention in more particular worthy to be mentioned are those compounds of formula I*,

in which

### R1 is methyl,

### R2 is methyl, chlorine, fluorine or methoxy,

### R3 is methyl, chlorine, fluorine or methoxy,

### R4 is hydrogen,

### X is oxygen, sulphur or NH,

### R5 is hydrogen,

and the salts of these compounds.

Compounds according to this invention to be emphasized are those compounds of formula I*,

in which

### R1 is methyl,

### R2 is methyl, chlorine, fluorine or methoxy,

### R3 is methyl, chlorine, fluorine or methoxy,

### R4 is hydrogen,

### X is NH,

### R5 is hydrogen,

and the salts of these compounds.

In a further embodiment (embodiment x) of this invention, compounds according to this invention include those compounds of formula I*

in which

### R1 is methyl or ethyl,

### R2 is halogen, trifluoromethyl, methoxy or ethoxy,

### R3 is halogen, trifluoromethyl, methoxy or ethoxy,

### R4 is hydrogen,

### X is oxygen, sulphur or NH,

### R5 is hydrogen,

and the salts of these compounds.

Compounds according to embodiment x of this invention more worthy to be mentioned are those compounds of formula I*,

in which

### R1 is methyl or ethyl,

### R2 is chlorine, fluorine, trifluoromethyl or methoxy,

### R3 is chlorine, fluorine, trifluoromethyl or methoxy,

### R4 is hydrogen,

### X is oxygen, sulphur or NH,

### R5 is hydrogen,

and the salts of these compounds.

Compounds according to embodiment x of this invention further more worthy to be mentioned are those compounds of formula I*,

in which

### R1 is methyl,

### R2 is chlorine, fluorine or methoxy,

### R3 is chlorine, fluorine or methoxy,

### R4 is hydrogen,

### X is oxygen, sulphur or NH,

### R5 is hydrogen,

and the salts of these compounds.

Compounds according to embodiment x of this invention in particular worthy to be mentioned are those compounds of formula I*,

in which

### R1 is methyl,

either

### R2 is methoxy, and

### R3 is methoxy,

or

### R2 is chlorine or fluorine, and

### R3 is chlorine or fluorine,

### R4 is hydrogen,

### X is oxygen, sulphur or NH,

### R5 is hydrogen,

and the salts of these compounds.

Compounds according to embodiment x of this invention in more particular worthy to be mentioned are those compounds of formula I*,

in which

### R1 is methyl,

### R2 is chlorine, fluorine or methoxy,

### R3 is chlorine, fluorine or methoxy,

### R4 is hydrogen,

### X is NH,

### R5 is hydrogen,

and the salts of these compounds.

In a yet further embodiment (embodiment y) of this invention, compounds according to this invention include those compounds of formula I*

in which

### R1 is methyl, ethyl or cyclopropyl,

### R2 is methyl, ethyl, halogen, trifluoromethyl, methoxy or ethoxy,

### R3 is methyl, ethyl, halogen, trifluoromethyl, methoxy or ethoxy,

### R4 is hydrogen,

### X is NH,

### R5 is methyl, ethyl, halogen, ethoxy or methoxy,

and the salts of these compounds.

Compounds according to embodiment y of this invention more worthy to be mentioned are those compounds of formula I*,

in which

### R1 is methyl or ethyl,

### R2 is methyl, chlorine, fluorine, trifluoromethyl or methoxy,

### R3 is methyl, chlorine, fluorine, trifluoromethyl or methoxy,

### R4 is hydrogen,

### X is NH,

### R5 is methyl, chlorine, fluorine or methoxy,

and the salts of these compounds.

Compounds according to embodiment y of this invention further more worthy to be mentioned are those compounds of formula I*,

in which

### R1 is methyl,

### R2 is methyl, chlorine, fluorine or methoxy,

### R3 is methyl, chlorine, fluorine or methoxy,

### R4 is hydrogen,

### X is NH,

### R5 is methyl, chlorine or fluorine,

and the salts of these compounds.

Compounds according to embodiment y of this invention in particular worthy to be mentioned are those compounds of formula I*,

in which

### R1 is methyl,

### R2 is methyl or methoxy,

### R3 is methyl or methoxy,

### R4 is hydrogen,

### X is NH,

### R5 is methyl, chlorine or fluorine,

and the salts of these compounds.

Further compounds according to embodiment y of this invention in particular worthy to be mentioned are those compounds of formula I*,

in which

### R1 is methyl,

### R2 is methyl,

### R3 is methyl,

### R4 is hydrogen,

### X is NH,

### R5 is methyl, chlorine or fluorine,

and the salts of these compounds.

Further compounds according to embodiment y of this invention in particular worthy to be mentioned are those compounds of formula I*,

in which

### R1 is methyl,

### R2 is methoxy,

### R3 is methoxy,

### R4 is hydrogen,

### X is NH,

### R5 is methyl, chlorine or fluorine,

and the salts of these compounds.

Compounds according to embodiment y of this invention in more particular worthy to be mentioned are those compounds of formula I*,

in which

### R1 is methyl,

### R2 is methyl or methoxy,

### R3 is methyl or methoxy,

### R4 is hydrogen,

### X is NH,

### R5 is methyl or fluorine,

and the salts of these compounds.

Further compounds according to embodiment y of this invention in more particular worthy to be mentioned are those compounds of formula I*,

in which

### R1 is methyl,

### R2 is methyl,

### R3 is methyl,

### R4 is hydrogen,

### X is NH,

### R5 is methyl or fluorine,

and the salts of these compounds.

Further compounds according to embodiment y of this invention in more particular worthy to be mentioned are those compounds of formula I*,

in which

### R1 is methyl,

### R2 is methoxy,

### R3 is methoxy,

### R4 is hydrogen,

### X is NH,

### R5 is methyl or fluorine,

and the salts of these compounds.

In a still yet further embodiment (embodiment z) of this invention, compounds according to this invention include those compounds of formula I*

in which

### R1 is methyl, ethyl or cyclopropyl,

### R2 is methyl or ethyl,

### R3 is methyl, ethyl, halogen, trifluoromethyl, methoxy or ethoxy,

### R4 is hydrogen,

### X is NH,

### R5 is hydrogen, methyl, ethyl, halogen, ethoxy or methoxy,

and the salts of these compounds.

Compounds according to embodiment z of this invention more worthy to be mentioned are those compounds of formula I*,

in which

### R1 is methyl or ethyl,

### R2 is methyl,

### R3 is methyl, chlorine, fluorine, trifluoromethyl or methoxy,

### R4 is hydrogen,

### X is NH,

### R5 is hydrogen, methyl, chlorine, fluorine or methoxy,

and the salts of these compounds.

Compounds according to embodiment z of this invention further more worthy to be mentioned are those compounds of formula I*,

in which

### R1 is methyl,

### R2 is methyl,

### R3 is methyl, chlorine, fluorine or methoxy,

### R4 is hydrogen,

### X is NH,

### R5 is hydrogen, methyl, chlorine or fluorine,

and the salts of these compounds.

Compounds according to embodiment z of this invention in particular worthy to be mentioned are those compounds of formula I*,

in which

### R1 is methyl,

### R2 is methyl,

### R3 is methyl or methoxy,

### R4 is hydrogen,

### X is NH,

### R5 is hydrogen, methyl, chlorine or fluorine,

and the salts of these compounds.

Compounds according to embodiment z of this invention in more particular worthy to be mentioned are those compounds of formula I*,

in which

### R1 is methyl,

### R2 is methyl,

### R3 is methyl or methoxy,

### R4 is hydrogen,

### X is NH,

### R5 is hydrogen, methyl or fluorine,

and the salts of these compounds.

As exemplary compounds according to this invention the following compounds of formula I*, in which

### R1 is methyl,

### R4 is hydrogen, and

### R5 is hydrogen,

and the salts thereof,
may be mentioned by means of the substituent meanings for X, R2 and R3 in the Table 1 given below.

As further exemplary compounds according to this invention the following compounds of formula I*, in which

### R1 is methyl,

### R4 is hydrogen, and

### R5 is bonded to the 5-position of the scaffold, and is fluorine,

and the salts thereof,
may be mentioned by means of the substituent meanings for X, R2 and R3 in the Table 1 given below.

As further exemplary compounds according to this invention the following compounds of formula I*, in which

### R1 is methyl,

### R4 is hydrogen, and

### R5 is bonded to the 7-position of the scaffold, and is fluorine,

and the salts thereof,
may be mentioned by means of the substituent meanings for X, R2 and R3 in the Table 1 given below.

As further exemplary compounds according to this invention the following compounds of formula I*, in which

### R1 is methyl,

### R4 is hydrogen, and

### R5 is bonded to the 5-position of the scaffold, and is methyl,

and the salts thereof,
may be mentioned by means of the substituent meanings for X, R2 and R3 in the Table 1 given below.

As further exemplary compounds according to this invention the following compounds of formula I*, in which

### R1 is methyl,

### R4 is hydrogen, and

### R5 is bonded to the 7-position of the scaffold, and is methyl,

and the salts thereof,
may be mentioned by means of the substituent meanings for X, R2 and R3 in the Table 1 given below.

TABLE 1

| | X | R2 | R3 |
|---|---|---|---|
| 1.) | NH | methoxy | methoxy |
| 2.) | O | methoxy | methoxy |
| 3.) | S | methoxy | methoxy |
| 4.) | NH | chlorine | chlorine |
| 5.) | O | chlorine | chlorine |
| 6.) | S | chlorine | chlorine |
| 7.) | NH | fluorine | fluorine |
| 8.) | O | fluorine | fluorine |
| 9.) | S | fluorine | fluorine |
| 10.) | NH | chlorine | methoxy |
| 11.) | O | chlorine | methoxy |
| 12.) | S | chlorine | methoxy |
| 13.) | NH | fluorine | methoxy |
| 14.) | O | fluorine | methoxy |
| 15.) | S | fluorine | methoxy |
| 16.) | NH | chlorine | fluorine |
| 17.) | O | chlorine | fluorine |
| 18.) | S | chlorine | fluorine |
| 19.) | NH | methyl | methyl |
| 20.) | O | methyl | methyl |
| 21.) | S | methyl | methyl |
| 22.) | NH | methyl | methoxy |
| 23.) | O | methyl | methoxy |
| 24.) | S | methyl | methoxy |
| 25.) | NH | methyl | chlorine |
| 26.) | O | methyl | chlorine |
| 27.) | S | methyl | chlorine |
| 28.) | NH | methyl | fluorine |
| 29.) | O | methyl | fluorine |
| 30.) | S | methyl | fluorine |

Particular exemplary compounds according to the present invention may include, without being restricted thereto, any compound selected from
(3aS,10R)-10-(3,5-Dimethoxy-phenyl)-2-methyl-1-thioxo-1,2,3a,4,9,10-hexahydro-2,9,10a-triaza-cyclopenta[b]fluoren-3-one
(3aS,10R)-10-(3,5-Dimethoxy-phenyl)-2-methyl-1-thioxo-1,2,4,10-tetrahydro-3aH-9-thia-2,10a-diaza-cyclopenta[b]fluoren-3-one
(3aS,10R)-10-(3,5-Dichloro-phenyl)-2-methyl-1-thioxo-1,2,3a,4,9,10-hexahydro-2,9,10a-triaza-cyclopenta[b]fluoren-3-one
(3aS,10R)-10-(3,5-Difluoro-phenyl)-2-methyl-1-thioxo-,2,3a,4,9,10-hexahydro-2,9,10a-triaza-cyclopenta[b]fluoren-3-one
(3aS,10R)-10-(3,5-Dimethyl-phenyl)-2-methyl-1-thioxo-1,2,3a,4,9,10-hexahydro-2,9,10a-triaza-cyclopenta[b]fluoren-3-one
(3aS,10R)-10-(3,5-Dimethoxy-phenyl)-7-fluoro-2-methyl-1-thioxo-1,2,3a,4,9,10-hexahydro-2,9,10a-triaza-cyclopenta[b]fluoren-3-one
(3aS,10R(-10-(3,5-Dimethoxy-phenyl)-2,5-dimethyl-1-thioxo-1,2,3a,4,9,10-hexahydro-2,9,10a-triaza-cyclopenta[b]fluoren-3-one and
(3aS,10R)-10(3,5-Dimethoxy-phenyl)-2,7-dimethyl-1-thioxo-1,2,3a,4,9,10-hexahydro-2,9,10a-triaza-cyclopenta[b]fluoren-3-one and the salts thereof.

A particular concern within the compounds according to this invention refers to those compounds of formula I*, in which X is NH.

A particular group within the compounds according to this invention refers to those compounds of formula I*, in which R5 is hydrogen.

Another particular group within the compounds according to this invention refers to those compounds of formula I*, in which X is NH, and R5 is hydrogen.

A special interest in the compounds according to this invention refers to those compounds of formula I which are included—within the scope of this invention—by one or, when possible, by more of the following special embodiments:

A special embodiment (embodiment 1) of the compounds of formula I according to this invention refers to those compounds of formula I, in which

### R1 is methyl.

Another special embodiment (embodiment 2) of the compounds of formula I according to this invention refers to those compounds of formula I, in which

### R1 is ethyl.

Another special embodiment (embodiment 3) of the compounds of formula I according to this invention refers to those compounds of formula I, in which

### X is oxygen.

Another special embodiment (embodiment 4) of the compounds of formula I according to this invention refers to those compounds of formula I, in which

### X is sulphur.

Another special embodiment (embodiment 5) of the compounds of formula I according to this invention refers to those compounds of formula I, in which

### X is NH.

Another special embodiment (embodiment 6) of the compounds of formula I according to this invention refers to those compounds of formula I, in which

### R2 is methoxy, chlorine or fluorine, and

### R3 is methoxy, chlorine or fluorine.

Another special embodiment (embodiment 7) of the compounds of formula I according to this invention refers to those compounds of formula I, in which

### R2 is methoxy, and

### R3 is methoxy.

Another special embodiment (embodiment 8) of the compounds of formula I according to this invention refers to those compounds of formula I, in which

### R2 is halogen, such as e.g. chlorine or fluorine, and

### R3 is halogen, such as e.g. chlorine or fluorine.

Another special embodiment (embodiment 9) of the compounds of formula I according to this invention refers to those compounds of formula I, in which

### R2 is chlorine, and

### R3 is chlorine.

Another special embodiment (embodiment 9) of the compounds of formula I according to this invention refers to those compounds of formula I, in which

### R2 is fluorine, and

### R3 is fluorine.

Another special embodiment (embodiment 10) of the compounds of formula I according to this invention refers to those compounds of formula I, in which

### R2 is methoxy.

Another special embodiment (embodiment 11) of the compounds of formula I according to this invention refers to those compounds of formula I, in which

### R3 is methoxy.

Another special embodiment (embodiment 12) of the compounds of formula I according to this invention refers to those compounds of formula I, in which

### R2 is fluorine.

Another special embodiment (embodiment 13) of the compounds of formula I according to this invention refers to those compounds of formula I, in which

### R3 is fluorine.

Another special embodiment (embodiment 14) of the compounds of formula I according to this invention refers to those compounds of formula I, in which

### R2 is chlorine.

Another special embodiment (embodiment 15) of the compounds of formula I according to this invention refers to those compounds of formula I, in which

### R3 is chlorine.

Another special embodiment (embodiment 16) of the compounds of formula I according to this invention refers to those compounds of formula I, in which

### R2 is methyl.

Another special embodiment (embodiment 17) of the compounds of formula I according to this invention refers to those compounds of formula I, in which

### R3 is methyl.

Another special embodiment (embodiment 18) of the compounds of formula I according to this invention refers to those compounds of formula I, in which

### R2 is methyl, and

### R3 is methoxy.

Another special embodiment (embodiment 19) of the compounds of formula I according to this invention refers to those compounds of formula I, in which

### R2 is methyl, and

### R3 is methyl.

Another special embodiment (embodiment 20) of the compounds of formula I according to this invention refers to those compounds of formula I, in which

### R5 is hydrogen.

Another special embodiment (embodiment 21) of the compounds of formula I according to this invention refers to those compounds of formula I, in which

### R5 is fluorine.

Another special embodiment (embodiment 22) of the compounds of formula I according to this invention refers to those compounds of formula I, in which

### R5 is methyl.

Another special embodiment (embodiment 23) of the compounds of formula I according to this invention refers to those compounds which are from formula I* as shown above.

Another special embodiment (embodiment 24) of the compounds according to the present invention refers to those compounds which are from formula I*, in which

### R4 is hydrogen, and

### R2, R3 and X have any of the meanings indicated in Table 1 given above.

Another special embodiment (embodiment 25) of the compounds according to the present invention refers to those compounds which are from formula I*, in which

### R1 is methyl,

### R4 is hydrogen, and

### R2, R3 and X have any of the meanings indicated in Table 1 given above.

It is to be understood that the present invention includes any or all possible combinations and subsets of the special embodiments defined hereinabove.

The compounds according to the invention can be prepared e.g. as described exemplarily as follows and according to the following specified reaction steps, or, particularly, in a manner as described by way of example in the following examples, or analogously or similarly thereto according to preparation procedures or synthesis strategies known to the person skilled in the art.

As shown in the synthesis route outlined in scheme 1 below, compounds of formula IV, in which X has the meanings given above, are condensed and cyclized in a Pictet-Spengler reaction with benzaldehydes of formula III, in which R2, R3 and R4 have the meanings mentioned above, to give the corresponding compounds of formulae IIa and/or IIb mostly as a mixture. Said Pictet-Spengler reaction can be carried out as it is known to the skilled person or as described in the following examples, advantageously in the presence of a suitable acid as a catalyst or promoter (e.g. trifluoroacetic acid) in a suitable solvent, for example toluene, at elevated temperature.

Compounds of formula IV, in which X and R5 have the meanings given above, are known, commercially available (such as e.g. certain tryptophane derivatives thereof) or can be prepared according to known procedures or may be accessible as described later, e.g. by esterification of corresponding free acid compounds which are known or obtainable in a known manner. Thus, e.g. (R)-2-amino-3-(benzothiophen-3-yl)-propionic acid methyl ester is obtained from D-thiotryptophan by esterification reaction. Said esterification reaction can be carried out in a manner habitual per se to the skilled person, e.g. via an appropriate corresponding activated form of the acid, such as, for example, the corresponding acid chloride—obtainable with the aid of thionyl chloride or the like—which is reacted with the corresponding alcohol, preferably methanol. D-Thiotryptophan is known or can be obtained in a known manner.

Compounds of formula III are known or can be obtained according to known procedures, for example by formylation of appropriate aromatic compounds, e.g. via hydroxymethylation and subsequent oxidation to the aldehyde, or by reduction of appropriate benzoic acid derivatives to the aldehyde.

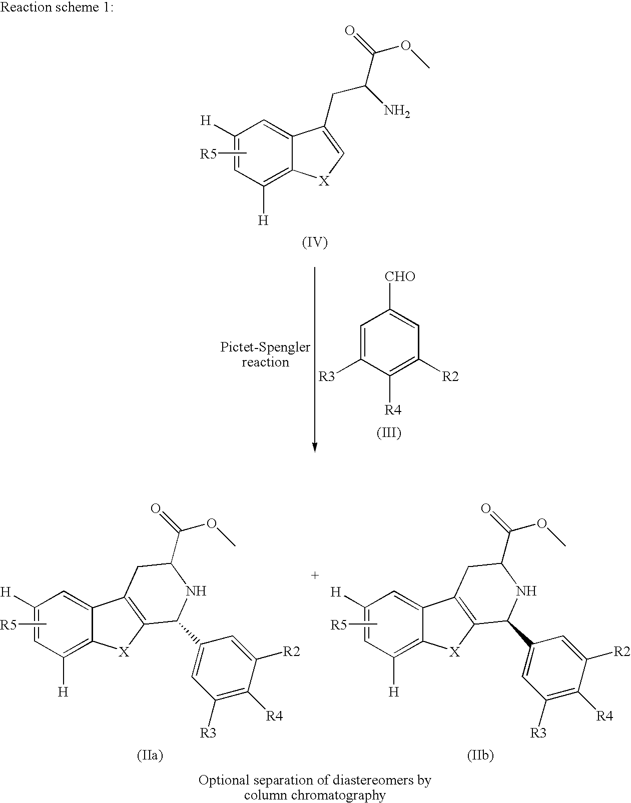

The compounds of formula IV can be employed in the abovementioned Pictet-Spengler reaction as racemate or enantiomerically pure compounds. Depending thereon, the mixture obtained can contain the compounds of formulae IIa and IIb as diastereomers or as diastereomeric racemates. Said mixture can be optionally separated in a manner habitual per se to the skilled person, such as, for example, diastereomeric compounds of formulae IIa and IIb can be separated e.g. by column chromatography.

If appropriate, said mixture can be also used in the next step without further separation of the diastereoisomers. Then, separation of diastereomers can be carried out subsequently to one of the following steps.

The abovementioned Pictet-Spengler reaction allows to prepare those compounds of formulae IIa or IIb in excess or diastereoselectively, in which the hydrogen atoms in positions 1 and 3 are located at the same side of the plane defined by the tetrahydropyridine ring.

When the compounds of formula IV are employed as racemic mixture in the abovementioned Pictet-Spengler reaction, the racemate comprising the enantiomeric compounds of formulae IIa′ and IIb′ may be obtained in excess or preferentially from said reaction.

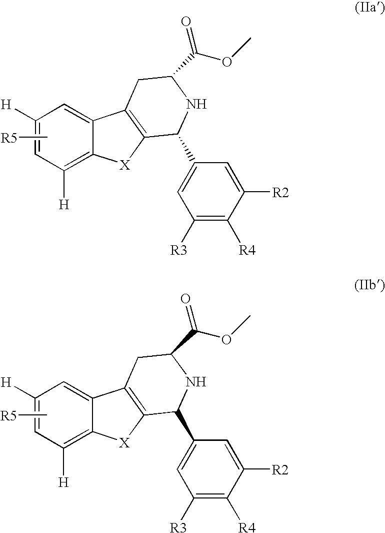

Starting from the appropriate pure enantiomers of the compounds of formula IV, corresponding compounds of either formula IIa′ or formula IIb′ (depending from the configuration of the starting compound of formula IV) can be obtained preferentially. Thus, e.g. when (R)-2-amino-3-(benzothiophen-3-yl)-propionic acid methyl ester is employed in the abovementioned Pictet-Spengler reaction, corresponding compounds of formula IIa′, in which X is S and R5 is hydrogen, are obtained preferentially.

Compounds of formula IIa′ or IIb′, e.g. in enantiomerically pure form or as racemic mixture or with corresponding diastereomers co-generated in the Pictet-Spengler reaction above, can be reacted with isothiocyanates of formula R1—N═C═S, in a thiohydantoin synthesis as shown in reaction scheme 2 to give the corresponding desired thiohydantoins of formula I* (from compounds of formula IIa′) or II* (from compounds of formula IIb′). Said thiohydantoin synthesis can be performed in an art-known manner or as described in the following examples, e.g. in the presence of microwaves.

Under the reaction conditions used in this thiohydantoin synthesis epimerization of the configuration of the chiral carbon atom 3a can be obtained.

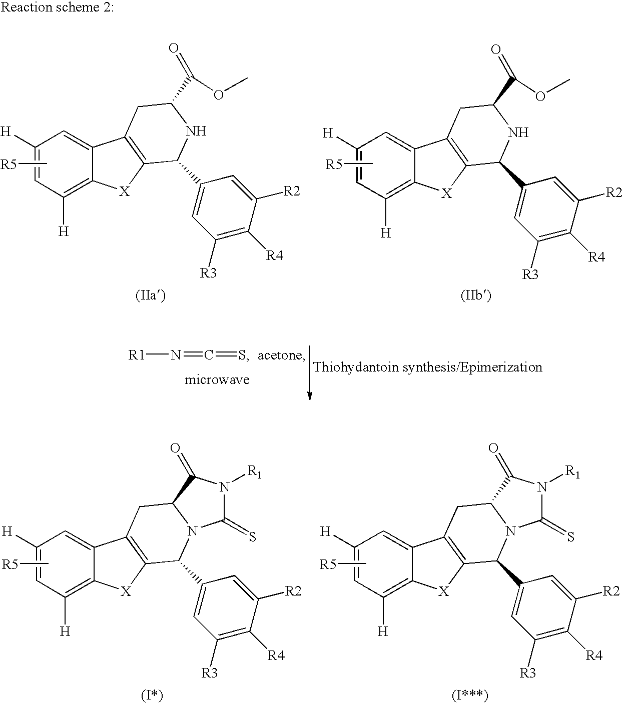

If necessary, the configuration of the chiral carbon atom 3a of compounds of formula I may be also epimerized via deprotonation/reprotonation with the aid of a suitable base such as e.g. potassium carbonate in a suitable solvent such as e.g. acetonitrile.

When the compounds of formulae I*, I**, I*** or I**** are obtained as racemic mixture, the corresponding enantiomerically pure compounds may be accessible by art-known separation techniques, such as e.g. those described above.

Optionally, compounds of the formula I can be converted into their salts, or, optionally, salts of the compounds of the formula I can be converted into the free compounds.

Compounds of formula IV, in which X and R5 have the meanings given above, may be accessible as shown in reaction scheme 3.

Starting from compounds of formula IX, in which X and R5 have the meanings mentioned above, and which are known from the art or accessible in an art-known manner, the corresponding 9-membered bicyclic derivatives of formula VIII can be obtained by cyclization reaction customary per se to the person skilled in the art (e.g. the synthesis of substituted benzothiophenes starting from well known substituted thio phenoles are described in Tsuri et al, J. Med. Chem. 2003, 46, 2446-2455). Depending on the substitution pattern of compounds of formula IX, the separation of the resulting region isomers might be necessary.

The corresponding chloro methyl derivative of formula VII can be prepared by art-known chloromethylation reaction, such as e.g. as described in Efange et al, J. Med. Chem., 1998, 41, 4486-4491 by the reaction of compounds of formula VIII with formaldehyde and HCl.

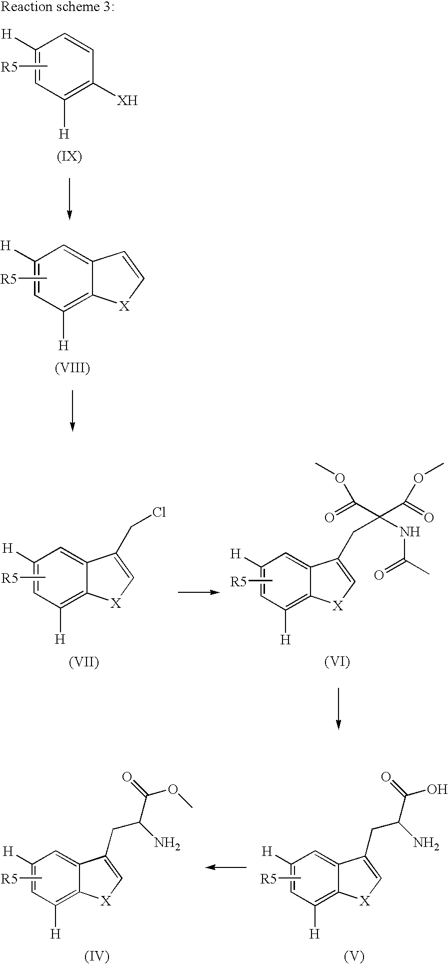

Compounds of formula VII can be converted into corresponding compounds of formula VI, which can be saponified and decarboxylated to give amino acids of formula V. These procedures are known from the art, such as e.g. described in Rao et al, International Journal of Peptide & Protein Research 1987, 29, 118-125, or can be carried out analogously or similarly to art-known procedures.

The amino acids of formula V can be converted into the corresponding ester (e.g. methyl ester) derivatives of formula IV in a manner habitual per se to the skilled person.

Enantiomerically pure starting compounds according to this invention (e.g. amino acids or amino acid derivatives, particularly tryptophans) can be obtained according to art-known processes, such as e.g. from the corresponding racemates as described above. Therefore enantiomerically pure amino acids or amino acid derivatives (e.g. ester derivatives) can be obtained, for example, by means of salt formation of the racemic compounds with optically active acids (such as e.g. tartaric acid, mandelic acid, camphorsulfonic acid or the like), subsequent resolution of the salts [e.g. by (fractional) crystallization from a suitable solvent] and release of the desired compound from the salt; by kinetic resolution of the racemic compounds, such as by enzymatic racemate resolution, e.g. during enzymatic saponification of the corresponding racemic amino acid esters using e.g. a suitable lipase; or by stereoselective amino acid synthesis, e.g. using the Schöllkopf bis-lactam ether chiral auxiliary; or by chromatographic separation of racemic compounds on chiral separating columns.

Thus, enantiomerically pure tryptophans can be obtained, for example, without being limited, as described in Tetrahedron Letters 40 (1999), 657-660 or in Chirality 8 (1996), 418-422, or analogously or similarly thereto.

It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesis” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000).

The substances according to the invention are isolated and purified in a manner known per se, for example by distilling off the solvent under reduced pressure and recrystallizing the residue obtained from a suitable solvent or subjecting it to one of the customary purification methods, such as, for example, column chromatography on a suitable support material.

Salts can be obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low-molecular-weight aliphatic alcohol, such as methanol, ethanol or isopropanol) which contains the desired acid or base, or to which the desired acid or base is then added. The salts can be obtained by filtering, reprecipitating, precipitating with a nonsolvent for the addition salt or by evaporating the solvent. Salts obtained can be converted into the free compounds, which can in turn be converted into salts, by alkalization or by acidification. In this manner, pharmacologically unacceptable salts can be converted into pharmacologically acceptable salts.

Suitably, the conversions mentioned in this invention can be carried out analogously or similarly to methods which are familiar per se to the person skilled in the art.

The person skilled in the art knows on the basis of his/her knowledge and on the basis of those synthesis routes, which are shown and described within the description of this invention, how to find other possible synthesis routes for compounds according to this invention. All these other possible synthesis routes are also part of this invention.

The present invention also relates to intermediates and methods useful in synthesizing compounds according to this invention.

Having described the invention in detail, the scope of the present invention is not limited only to those described characteristics or embodiments. As will be apparent to persons skilled in the art, modifications, analogies, variations, derivations, homologisations and adaptations to the described invention can be made on the base of art-known knowledge and/or, particularly, on the base of the disclosure (e.g. the explicite, implicite or inherent disclosure) of the present invention without departing from the spirit and scope of this invention as defined by the scope of the appended claims.

The following examples serve to illustrate the invention further without restricting it. Likewise, further compounds according to this invention, whose preparation is not explicitly described, can be prepared in an analogous or similar manner or in a manner familiar per se to the person skilled in the art using customary process techniques.

The compounds of formula I according to the present invention which are mentioned as final compounds in the following examples, as well as the salts, stereoisomers and salts of the stereoisomers thereof, are a preferred subject of the present invention.

In the examples, m.p. stands for melting point, h for hour(s), min for minutes, conc. for concentrated, calc. for calculated, fnd. for found, EF for elemental formula, MS for mass spectrometry, M for molecular ion in mass spectrometry, and other abbreviations have their meanings customary per se to the skilled person.

According to common practice in stereochemistry, the symbols RS and SR are used to denote the specific configuration of each of the indicated chiral centers of a racemate. In more detail, for example, the term “(3aSR,10RS)” stands for a racemate comprising the one enantiomer having the configuration (3aS,10R) and the other enantiomer having the configuration (3aR,10S); each of these enantiomers in pure form as well as their mixtures including the racemic mixtures is part of this invention, whereby this enantiomer having the configuration (3aS,10R) is a preferred part of this invention.

### EXAMPLES

### Final Compounds

### 1. (3aS,10R)-10-(3,5-Dimethoxy-phenyl)-2-methyl-1-thioxo-1,2,3a,4,9,10-hexahydro-2,9,10a-triaza-cyclopenta[b]fluoren-3-one

To a solution of 200 mg (1R,3R)-1-(3,5-dimethoxy-phenyl)-2,3,4,9-tetrahydro-1H-β-carboline-3-carboxylic acid methyl ester (compound A1) in 5.00 ml acetone are added 300.0 mg methyl isothiocyanate. The mixture is heated to 150° C. for 15 min using a microwave reactor. The solvent is removed at reduced pressure and the residue is dissolved in ethyl acetate. The solution is washed with water. The organic layer is dried with magnesium sulfate and the solvent is removed at reduced pressure. After column chromatography (silica gel, toluene/ethyl acetate: 20:1) and crystallization from diisopropyl ether, 151 mg (68%) of the title compound is obtained as a colourless solid.

EF: C22 H21 N3 O3 S (407.49); fnd.: MS: m/z (MH⁺)=408.1

Starting from the appropriate ester compounds, which are mentioned or described explicitly below (compound A1), or which can be prepared in a manner known to the person skilled in the art analogously or similarly to the examples described herein and the appropriate isothiocyanate derivatives, the following compounds are obtained according to the procedure as in Example 1.

### 2. (3aSR,10RS)-10-(3,5-Dimethoxy-phenyl)-2-methyl-1-thioxo-1,2,4,10-tetrahydro-3aH-9-thia-2,10a-diaza-cyclopenta[b]fluoren-3-one

M.p.: 247-249° C.

C22 H2O N2 O3 S2 (424.54); fnd.: MS: m/z (MH⁺)=425.1

### 3. (3aS,10R)-10-(3,5-Dichloro-phenyl)-2-methyl-1-thioxo-1,2,3a,4,9,10-hexahydro-2,9,10a-triaza-cyclopenta[b]fluoren-3-one

M.p.: 277° C.

C20 H15 C12 N3 O S (416.33); fnd.: MS: m/z (MH⁺)=416.0

### 4. (3aS,10R)-10-(3,5-Difluoro-phenyl)-2-methyl-1-thioxo-1,2,3a,4,9,10-hexahydro-2,9,10a-triaza-cyclopenta[b]fluoren-3-one

M.p.: 308-310° C.

C20 H15 F2N3 O S (383.42); fnd.: MS: m/z (MH⁺)=384.1

### 5. (3aS,10R)-10-(3,5-Dimethyl-phenyl)-2-methyl-1-thioxo-1,2,3a,4,9,10-hexahydro-2,9,10a-triaza-cyclopenta[b]fluoren-3-one

To a solution of 300 mg (1R,3R)-1-(3,5-dimethyl-phenyl)-2,3,4,9-tetrahydro-1H-β-carboline-3-carboxylic acid methyl ester (compound A2) in 4.50 ml acetone are added 280.0 mg methyl isothiocyanate. The mixture is heated to 150° C. for 10 min using a microwave reactor. The solvent is removed at reduced pressure and the residue is dissolved in ethyl acetate. The solution is washed with water. The organic layer is dried with magnesium sulfate and the solvent is removed at reduced pressure. After column chromatography (silica gel, toluene) 305 mg of the title compound is obtained as a colourless solid

EF: C22 H21 N3 O S (375.50); fnd.: MS: m/z (MH⁺)=376.1

M.p.: 252-257° C.

Starting from the appropriate ester compounds, which are mentioned or described explicitly below, or which can be prepared in a manner known to the person skilled in the art analogously or similarly to the examples described herein, the following compounds are obtained according to the procedure as in Example 5.

### 6. (3aSR,10RS)-10-(3,5-Dimethoxy-phenyl)-7-fluoro-2-methyl-1-thioxo-1,2,3a,4,9,10-hexahydro-2,9,10a-triaza-cyclopenta[b]fluoren-3-one

M.p.: 199-202° C.

C22 H20 F N3 O3 S (425.49)); fnd.: MS: m/z (MH⁺)=426.1

### 7. (3aSR,10RS)-10-(3,5-Dimethoxy-phenyl)-2,5-dimethyl-1-thioxo-1,2,3a,4,9,10-hexahydro-2,9,10a-triaza-cyclopenta[b]fluoren-3-one

M.p.: 279-291° C.

C23 H23 N3 O3S (421.52); fnd.: MS: m/z (MH⁺)=422.0

### 8. (3aSR,10RS)-10-(3,5-Dimethoxy-phenyl)-2,7-dimethyl-1-thioxo-1,2,3a,4,9,10-hexahydro-2,9,10a-triaza-cyclopenta[b]fluoren-3-one

M.p.: 216-218° C.

C23 H23 N3 O3S (421.52); fnd.: MS: m/z (MH⁺)=422.1

### Starting Compounds

### A1. (1R,3R)-1-(3,5-Dimethoxy-phenyl)-2,3,4,9-tetrahydro-1H-β-carboline-3-carboxylic acid methyl ester

To solution of 2.20 g D-tryptophan methyl ester in 25 ml dichloro methane are added 1.87 g 3,5-dimethoxy benzaldehyde and 4.6 ml trimethyl orthoformate. The solution is stirred at room temperature over night. The solvent is removed at reduced pressure. The residue is dissolved in 45 ml and cooled to 0° C. 1.40 ml trifluoro acetic acid are added dropwise. After 90 min at 0° C. the mixture was stirred for additional 48 h at room temperature.

The solution is washed with a saturated aqueous solution of sodium carbonate. The organic layer is washed with brine and dried with magnesium sulfate. The solvent is removed at reduced pressure. The title compound is isolated by column chromatography (silica gel, ethyl acetate/hexane: 3/7). 1.06 g (34%) are obtained as a pale yellow, highly viscous oil.

### A2. (1R,3R)-1-(3,5-Dimethyl-phenyl)-2,3,4,9-tetrahydro-1H-β-carboline-3-carboxylic acid methyl ester

To solution of 2.41 g D-tryptophan methyl ester, 2.03 g 3,5-dimethyl benzaldehyde are added at room temperature 7.93 g trifluoro acetic acid. The solution is heated to 40° C. for 3 hours and stirred at room temperature over night.

Water is added to the reaction mixture and the solution is made basic adding a saturated aqueous solution of sodium carbonate. Ethyl acetate is added, the organic layer is washed with water and dried with magnesium sulfate. The solvent is removed at reduced pressure. After column chromatography 1.49 g of the title compound are obtained as an oil.

Starting from the appropriate art-known or commercially available (thio)tryptophan derivatives and benzaldehyde derivatives the following and further relevant, non-explicitly described starting compounds are obtained analogously or similarly to the procedure as to attain to Example A1 or A2.

### A3. (1R,3R)-1-(3,5-Dichloro-phenyl)-2,3,4,9-tetrahydro-1H-β-carboline-3-carboxylic acid methyl ester

### A4. (1R,3R)-1-(3,5-Difluoro-phenyl)-2,3,4,9-tetrahydro-1H-β-carboline-3-carboxylic acid methyl ester

### B1. D-Tryptophan methyl ester

The title compound can be obtained by methylesterificaton of D-tryptophan in methanol with the aid of thionylchloride according to standard procedures.

### Commercial Utility

The compounds according to the present invention have miscellaneous valuable pharmacological properties which can make them commercially applicable.

The compounds according to the invention therefore can be employed as therapeutic agents for the treatment and prophylaxis of diseases in human and veterinary medicine.

Thus, for example, in more embodimental detail, the compounds according to this invention are potent and highly efficacious cell-cycle specific inhibitors of cellular (hyper)proliferation and/or inducers of apoptosis in cancer cells. Therefore, these compounds are expected to be useful for treating (hyper)proliferative diseases and/or disorders responsive to the induction of apoptosis, in particular cancer.

Further on, these compounds can be useful in the treatment of benign or malignant neoplasia.

A “neoplasia” is defined by cells displaying aberrant cell proliferation and/or survival and/or a block in differentiation. A “benign neoplasia” is described by hyperproliferation of cells, incapable of forming an aggressive, metastasizing tumor in-vivo. In contrast, a “malignant neoplasia” is described by cells with multiple cellular and biochemical abnormalities, capable of forming a systemic disease, for example forming tumor metastasis in distant organs.

Various diseases are caused by limitless replicative potential and aberrant cell proliferation (“hyperproliferation”) as well as evasion from apoptosis. These diseases include e.g. benign hypoplasia like that of the prostate (“BPH”) or colon epithelium, psoriasis, glomerulonephritis or osteoarthritis. Most importantly these diseases include malignant neoplasia commonly described as cancer and characterized by tumor cells finally metastasizing into distinct organs or tissues. Malignant neoplasia include solid and hematological tumors. Solid tumors are exemplified by tumors of the breast, bladder, bone, brain, central and peripheral nervous system, colon, endocrine glands (eg thyroid and adrenal cortex), esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva. Malignant neoplasia include inherited cancers exemplified by retinoblastoma and Wilms tumor. In addition, malignant neoplasia include primary tumors in said organs and corresponding secondary tumors in distant organs (“tumor metastases”). Hematological tumors are exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, cancers of unknown primary site as well as AIDS related malignancies.

It is to be noted that a cancer disease as well as a malignant neoplasia does not necessarily require the formation of metastases in distant organs. Certain tumors exert devastating effects on the primary organ itself through their aggressive growth properties. These can lead to the destruction of the tissue and organ structure finally resulting in failure of the assigned organ function.

Neoplastic cell proliferation might effect normal cell behaviour and organ function. For example the formation of new blood vessels, a process described as neovascularization, is induced by tumors or tumor metastases. Compounds according to this invention can be commercially applicable for treatment of pathophysiological relevant processes caused by benign or neoplastic cell proliferation, such as but not limited to neovascularization by unphysiological proliferation of vascular endothelial cells.

Drug resistance is of particular importance for the frequent failure of standard cancer therapeutics. This drug resistance is caused by various cellular and molecular mechanisms like overexpression of drug efflux pumps or mutation within the cellular target protein. The commercial applicability of the compounds according to this invention is not limited to 1^{st} line treatment of patients. Patients with resistance to defined cancer chemotherapeutics or target specific anti-cancer drugs (2^{nd} or 3^{rd} line treatment) can be also amenable for treatment with the compounds according to this invention.

Further on, a special interest in the compounds according to the present invention lies in their potency to combat (hyper)proliferative diseases and/or disorders responsive to the induction of apoptosis, in particular cancer, without substantially inhibiting mitotic kinesin spindle protein (KSP/Eg5).

A particular interest in some compounds according to the present invention lies in their intrinsic potency to destabilize and/or inhibit tubuline polymerization in vitro. Furthermore, direct tubuline binding, as shown by competitive colchicine displacement on tubuline, could be deduced for some compounds according to this invention. Compounds fulfilling these characteristics may interfere with cellular microtubule dynamic instability and as a consequence irreversibly disturb the mitotic process, which limits or stops cellular (hyper)proliferation and may ultimately lead to cell death.

Compounds according to the present invention can be commercially applicable for treatment, prevention or amelioration of the diseases of benign and malignant behavior as described before, such as e.g. benign or malignant neoplasia, particularly cancer, such as e.g. any of those cancer diseases described above.

In the context of their properties, functions and usabilities mentioned herein, the compounds according to the present invention are expected to be distinguished by valuable and desirable effects related therewith, such as e.g. by low toxicity, superior bioavailability in general (such as e.g. good enteral absorption), superior therapeutic window, absence of significant side effects, and/or further beneficial effects related with their therapeutic and pharmaceutical suitability.

The invention further includes a method for treating (hyper)proliferative diseases and/or disorders responsive to the induction of apoptosis, particularly those diseases, disorders, conditions or illnesses mentioned above, in mammals, including humans, suffering therefrom comprising administering to said mammals in need thereof a pharmacologically active and therapeutically effective and tolerable amount of one or more of the compounds according to this invention.

The present invention further includes a method useful to modulate apoptosis and/or aberrant cell growth in the therapy of benign or malignant neoplastic diseases, such as e.g. cancer, comprising administering to a subject in need of such therapy a therapeutically active and pharmacologically effective and tolerable amount of one or more of the compounds according to this invention.

The present invention further relates to the use of the compounds according to this invention for the production of pharmaceutical compositions which are employed for the treatment, prophylaxis and/or amelioration of the illnesses mentioned.

The present invention further relates to the use of the compounds according to this invention for the production of pharmaceutical compositions which can be used in the treatment, prevention or amelioration of (hyper)proliferative diseases of benign or malignant behaviour and/or disorders responsive to the induction of apoptosis in a mammal, such as, for example, benign or malignant neoplasia, e.g. cancer.

The present invention further relates to the use of the compounds according to this invention for the production of pharmaceutical compositions which can be used use in the treatment, prevention or amelioration of disorders responsive to arresting of aberrant cell growth and/or induction of apoptosis.

The present invention further relates to the use of the compounds according to this invention for the production of pharmaceutical compositions for treating, preventing or ameliorating benign or malignant neoplasia, particularly cancer, such as e.g. any of those cancer diseases described above.

The present invention further relates to pharmaceutical compositions comprising one or more of the compounds according to this invention and a pharmaceutically acceptable carrier or diluent.

The present invention further relates to pharmaceutical compositions made by combining one or more of the compounds according to this invention and a pharmaceutically acceptable carrier or diluent.

The present invention further relates to pharmaceutical compositions comprising one or more of the compounds according to this invention and pharmaceutically acceptable auxiliaries and/or excipients.

The present invention further relates to combinations comprising one or more of the compounds according to this invention and pharmaceutically acceptable auxiliaries, excipients and/or vehicles, e.g. for treating, preventing or ameliorating benign or malignant neoplasia, particularly cancer, such as e.g. any of those cancer diseases described above.

The present invention further relates to a combination comprising a compound according to this invention and a pharmaceutically acceptable excipient, carrier and/or diluent, e.g. for treating, preventing or ameliorating benign or malignant neoplasia, particularly cancer, such as e.g. any of those cancer diseases described above.

The present invention further relates to a composition consisting essentially of a therapeutically effective and tolerable amount of one or more compounds according to this invention together with the usual pharmaceutically acceptable vehicles, diluents and/or excipients for use in therapy, e.g. for treating, preventing or ameliorating hyperproliferative diseases, such as e.g. cancer, and/or disorders responsive to induction of apoptosis.

The present invention further relates to compounds according to this invention for use in therapy, such as, for example, in the treatment, prevention or amelioration (hyper)proliferative diseases of benign or malignant behaviour and/or disorders responsive to the induction of apoptosis, such as e.g. those diseases mentioned herein, particularly cancer.

The present invention further relates to compounds according to this invention having anti-proliferative and/or apoptosis inducing activity.

The present invention further relates to pharmaceutical compositions according to this invention having anti-proliferative activity.

The present invention further relates to pharmaceutical compositions according to this invention having apoptosis inducing activity.

The invention further relates to the use of a pharmaceutical composition comprising one or more of the compounds according to this invention as sole active ingredient(s) and a pharmaceutically acceptable carrier or diluent in the manufacture of pharmaceutical products for the treatment and/or prophylaxis of the illnesses mentioned above.

Additionally, the invention relates to an article of manufacture, which comprises packaging material and a pharmaceutical agent contained within said packaging material, wherein the pharmaceutical agent is therapeutically effective inhibiting cellular (hyper)proliferation and/or inducing apoptosis, ameliorating the symptoms of a (hyper)proliferative disease and/or a disorder responsive to the induction of apoptosis, and wherein the packaging material comprises a label or package insert which indicates that the pharmaceutical agent is useful for preventing or treating a (hyper)proliferative disease and/or a disorder responsive to the induction of apoptosis, and wherein said pharmaceutical agent comprises one or more compounds according to the invention. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the compounds of the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the compounds, pharmaceutical compositions or combinations according to the invention may be performed in any of the generally accepted modes of administration available in the art. Illustrative examples of suitable modes of administration include intravenous, oral, nasal, parenteral, topical, transdermal and rectal delivery. Oral and intravenous delivery are preferred.

For the treatment of dermatoses, the compounds of the invention can be in particular administered in the form of those pharmaceutical compositions which are suitable for topical application. For the production of the pharmaceutical compositions, the compounds of the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions.

The pharmaceutical compositions according to the invention are prepared by processes known per se. The dosage of the compounds of the invention (=active compounds) is carried out in the order of magnitude customary for inhibitors of cellular (hyper)proliferation or apoptosis inducers. Topical application forms (such as ointments) for the treatment of dermatoses thus contain the active compounds in a concentration of, for example, 0.1-99%. The customary dose in the case of systemic therapy (p.o.) may be between 0.03 and 60 mg/kg per day, (i. v.) may be between 0.03 and 60 mg/kg/h. In another embodiment, the customary dose in the case of systemic therapy (p.o.) is between 0.3 and 30 mg/kg per day, (I. v.) is between 0.3 and 30 mg/kg/h.

The choice of the optimal dosage regime and duration of medication, particularly the optimal dose and manner of administration of the active compounds necessary in each case can be determined by a person skilled in the art on the basis of his/her expert knowledge.

Depending upon the particular disease, to be treated or prevented, additional therapeutic active agents, which are normally administered to treat or prevent that disease, may optionally be coadministered with the compounds according to this invention. As used herein, additional therapeutic agents that are normally administered to treat or prevent a particular disease are known as appropriate for the disease being treated.

For example, compounds according to this invention may be combined with one or more standard therapeutic agents used for treatment of the diseases as mentioned before.

In one particular embodiment, compounds according to this invention may be combined with one or more art-known anti-cancer agents, such as e.g. with one or more chemotherapeutic and/or target specific anti-cancer agents as described below.

Examples of known chemotherapeutic anti-cancer agents frequently used in combination therapy include, but not are limited to (i) alkylating/carbamylating agents such as Cyclophosphamid (Endoxan®), Ifosfamide (Holoxan®), Thiotepa (Thiotepa Lederle®), Melphalan (Alkeran®), or chloroethylnitrosurea (BCNU); (ii) platinum derivatives like cis-platen (Platinex® BMS), oxaliplatin or carboplatin (Cabroplat® BMS); (iii) antimitotic agents/tubulin inhibitors such as vinca alkaloids (vincristine, vinblastine, vinorelbine), taxanes such as Paclitaxel (Taxol®), Docetaxel (Taxotere®) and analogs as well as new formulations and conjugates thereof, epothilones such as Epothilone B (Patupilone®), Azaepothilone (Ixabepilone®) or ZK-EPO, a fully synthetic epothilone B analog; (iv) topoisomerase inhibitors such as anthracyclines (exemplified by Doxorubicin/Adriblastin®), epipodophyllotoxines (exemplified by Etoposide/Etopophos®) and camptothecin and camptothecin analogs (exemplified by Irinotecan/Camptosar® or Topotecan/Hycamtin®); (v) pyrimidine antagonists such as 5-fluorouracil (5-FU), Capecitabine (Xeloda®), Arabinosylcytosine/Cytarabin (Alexan®) or Gemcitabine (Gemzar®); (vi) purin antagonists such as 6mercaptopurine (Puri-Nethol®), 6-thioguanine or fludarabine (Fludara®) and finally (vii) folic acid antagonists such as methotrexate (Farmitrexat®) or premetrexed (Alimta®).

Examples of target specific anti-cancer drug classes used in experimental or standard cancer therapy include but are not limited to (i) kinase inhibitors such as e.g. Imatnib (Glivec®), ZD-1839/Geftinib (Iressa®), Bay43-9006 (Sorafenib), SU11248/Sunitinib (Sutent®) or OSI-774/Erlotinib (Tarceva®); (ii) proteasome inhibitors such as PS-341/Bortezumib (Velcade®); (iii) histone deacetylase inhibitors like SAHA, PXD101, MS275, MGCD0103, Depsipeptide/FK228, NVP-LBH589, NVP-LAQ824, Valproic acid (VPA) and butyrates (iv) heat shock protein 90 inhibitors like 17-allylaminogeldanamycin (17-AAG); (v) vascular targeting agents (VTAs) like combretastin A4 phosphate or AVE8062/AC7700 and anti-angiogenic drugs like the VEGF antibodies, such as Bevacizumab (Avastin®), or KDR tyrosine kinase inhibitors such as PTK787/ZK222584 (Vatalanib); (vi) monoclonal antibodies such as Trastuzumab (Herceptin®) or Rituximab (MabThera/Rituxan®) or Alemtuzumab (Campath®) or Tositumab (Bexxar®) or C225/Cetuximab (Erbitux®) or Avastin (see above) as well as mutants and conjugates of monoclonal antibodies, e.g. Gemtuzumab ozogamicin (Mylotarg®) or Ibritumomab tiuxetan (Zevalin®), and antibody fragments; (vii) oligonucleotide based therapeutics like G-3139/Oblimersen (Genasense®); (viii) Toll-like receptor/TLR 9 agonists like Promune®, TLR 7 agonists like Imiquimod (Aldara®) or Isatoribine and analogues thereof, or TLR 7/8 agonists like Resiquimod as well as immunostimulatory RNA as TLR 7/8 agonists; (ix) protease inhibitors (x) hormonal therapeutics such as anti-estrogens (e.g. Tamoxifen or Raloxifen), anti-androgens (e.g. Flutamide or Casodex), LHRH analogs (e.g. Leuprolide, Goserelin or Triptorelin) and aromatase inhibitors.

Other known target specific anti-cancer agents which may be used for combination therapy include bleomycin, retinoids such as all-trans retinoic acid (ATRA), DNA methyltransferase inhibitors such as the 2-deoxycytidine derivative Decitabine (Docagen®) and 5-Azacytidine, alanosine, cytokines such as interleukin-2, interferons such as interferon α2 or interferon-γ, death receptor agonists, such as TRAIL, DR4/5 agonistic antibodies, FasL and TNF-R agonists.

As exemplary anti-cancer agents, which may be useful in the combination therapy according to the present invention, any of the following drugs may be mentioned, without being restricted thereto, 5 FU, actinomycin D, ABARELIX, ABCIXIMAB, ACLARUBICIN, ADAPALENE, ALEMTUZUMAB, ALTRETAMINE, AMINOGLUTETHIMIDE, AMIPRILOSE, AMRUBICIN, ANASTROZOLE, ANCITABINE, ARTEMISININ, AZATHIOPRINE, BASILIXIMAB, BENDAMUSTINE, BEVACIZUMAB, BEXXAR, BICALUTAMIDE, BLEOMYCIN, BORTEZOMIB, BROXURIDINE, BUSULFAN, CAMPATH, CAPECITABINE, CARBOPLATIN, CARBOQUONE, CARMUSTINE, CETRORELIX, CHLORAMBUCIL, CHLORMETHINE, CISPLATIN, CLADRIBINE, CLOMIFENE, CYCLOPHOSPHAMIDE, DACARBAZINE, DACLIZUMAB, DACTINOMYCIN, DAUNORUBICIN, DECITABINE, DESLORELIN, DEXRAZOXANE, DOCETAXEL, DOXIFLURIDINE, DOXORUBICIN, DROLOXIFENE, DROSTANOLONE, EDELFOSINE, EFLORNITHINE, EMITEFUR, EPIRUBICIN, EPITIOSTANOL, EPTAPLATIN, ERBITUX, ERLOTINIB, ESTRAMUSTINE, ETOPOSIDE, EXEMESTANE, FADROZOLE, FINASTERIDE, FLOXURIDINE, FLUCYTOSINE, FLUDARABINE, FLUOROURACIL, FLUTAMIDE, FORMESTANE, FOSCARNET, FOSFESTROL, FOTEMUSTINE, FULVESTRANT, GEFITINIB, GENASENSE, GEMCITABINE, GLIVEC, GOSERELIN, GUSPERIMUS, HERCEPTIN, IDARUBICIN, IDOXURIDINE, IFOSFAMIDE, IMATINIB, IMPROSULFAN, INFLIXIMAB, IRINOTECAN, IXABEPILONE, LANREOTIDE, LETROZOLE, LEUPRORELIN, LOBAPLATIN, LOMUSTINE, LUPROLIDE, MELPHALAN, MERCAPTOPURINE, METHOTREXATE, METUREDEPA, MIBOPLATIN, MIFEPRISTONE, MILTEFOSINE, MIRIMOSTIM, MITOGUAZONE, MITOLACTOL, MITOMYCIN, MITOXANTRONE, MIZORIBINE, MOTEXAFIN, MYLOTARG, NARTOGRASTIM, NEBAZUMAB, NEDAPLATIN, NILUTAMIDE, NIMUSTINE, OCTREOTIDE, ORMELOXIFENE, OXALIPLATIN, PACLITAXEL, PALIVIZUMAB, PATUPILONE, PEGASPARGASE, PEGFILGRASTIM, PEMETREXED, PENTETREOTIDE, PENTOSTATIN, PERFOSFAMIDE, PIPOSULFAN, PIRARUBICIN, PLICAMYCIN, PREDNIMUSTINE, PROCARBAZINE, PROPAGERMANIUM, PROSPIDIUM CHLORIDE, RALOXIFEN, RALTITREXED, RANIMUSTINE, RANPIRNASE, RASBURICASE, RAZOXANE, RITUXIMAB, RIFAMPICIN, RITROSULFAN, ROMURTIDE, RUBOXISTAURIN, SARGRAMOSTIM, SATRAPLATIN, SIROLIMUS, SOBUZOXANE, SORAFENIB, SPIROMUSTINE, STREPTOZOCIN, SUNITINIB, TAMOXIFEN, TASONERMIN, TEGAFUR, TEMOPORFIN, TEMOZOLOMIDE, TENIPOSIDE, TESTOLACTONE, THIOTEPA, THYMALFASIN, TIAMIPRINE, TOPOTECAN, TOREMIFENE, TRAIL, TRASTUZUMAB, TREOSULFAN, TRIAZIQUONE, TRIMETREXATE, TRIPTORELIN, TROFOSFAMIDE, UREDEPA, VALRUBICIN, VATALANIB, VERTEPORFIN, VINBLASTINE, VINCRISTINE, VINDESINE, VINORELBINE, VOROZOLE and ZEVALIN.

The anti-cancer agents mentioned herein above as combination partners of the compounds according to this invention are meant to include pharmaceutically acceptable derivatives thereof, such as e.g. their pharmaceutically acceptable salts.

The person skilled in the art is aware on the base of his/her expert knowledge of the kind, total daily dosage(s) and administration form(s) of the additional therapeutic agent(s) coadministered. Said total daily dosage(s) can vary within a wide range.

In practicing the present invention, the compounds according to this invention may be administered in combination therapy separately, sequentially, simultaneously, concurrently or chronologically staggered (such as e.g. as combined unit dosage forms, as separate unit dosage forms, as adjacent discrete unit dosage forms, as fixed or non-fixed combinations, as kit-of-parts or as admixtures) with one or more standard therapeutics (chemotherapeutic and/or target specific anti-cancer agents), in particular art-known anti-cancer agents, such as e.g. any of those mentioned above.

In this context, the present invention further relates to a combination comprising

a first active ingredient, which is at least one compound according to this invention, and
a second active ingredient, which is at least one art-known anti-cancer agent, such as e.g. one or more of those mentioned herein above,
for separate, sequential, simultaneous, concurrent or chronologically staggered use in therapy, such as e.g. in therapy of any of those diseases mentioned herein.

The term “combination” according to this invention may be present as a fixed combination, a non-fixed combination or a kit-of-parts.

A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously, concurrently or chronologically staggered.

The present invention further relates to a pharmaceutical composition comprising a first active ingredient, which is at least one compound according to this invention, and a second active ingredient, which is at least one art-known anti-cancer agent, such as e.g. one or more of those mentioned herein above, and, optionally,

a pharmaceutically acceptable carrier or diluent,
for separate, sequential, simultaneous, concurrent or chronologically staggered use in therapy.

The present invention further relates to a combination product comprising

a.) at least one compound according to this invention formulated with a pharmaceutically acceptable carrier or diluent, and
b.) at least one art-known ant-cancer agent, such as e.g. one or more of those mentioned herein above, formulated with a pharmaceutically acceptable carrier or diluent.

The present invention further relates to a kit-of-parts comprising a preparation of a first active ingredient, which is a compound according to this invention, and a pharmaceutically acceptable carrier or diluent; a preparation of a second active ingredient, which is an art-known anti-cancer agent, such as one of those mentioned above, and a pharmaceutically acceptable carrier or diluent; for simultaneous, concurrent, sequential, separate or chronologically staggered use in therapy. Optionally, said kit comprises instructions for its use in therapy, e.g. to treat (hyper)proliferative diseases and/or disorders responsive to the induction of apoptosis, such as e.g. cancer, more precisely, any of those cancer diseases described above.

The present invention further relates to a combined preparation comprising at least one compound according to this invention and at least one art-known ant-cancer agent for simultaneous, concurrent, sequential or separate administration.

In this connection, the present invention further relates to combinations, compositions, formulations, preparations or kits according to the present invention having anti-proliferative and/or apoptosis inducing properties.

In addition, the present invention further relates to a method for treating in combination therapy (hyper)proliferative diseases and/or disorders responsive to the induction of apoptosis, such as e.g. cancer, in a patient comprising administering a combination, composition, formulation, preparation or kit as described herein to said patient in need thereof.

In addition, the present invention further relates to a method for treating (hyper)proliferative diseases of benign or malignant behaviour and/or disorders responsive to the induction of apoptosis, such as e.g. cancer, in a patient comprising administering in combination therapy separately, simultaneously, concurrently, sequentially or chronologically staggered a pharmaceutically active and therapeutically effective and tolerable amount of a pharmaceutical composition, which comprises a compound according to this invention and a pharmaceutically acceptable carrier or diluent, and a pharmaceutically active and therapeutically effective and tolerable amount of one or more art-known anti-cancer agents, such as e.g. one or more of those mentioned herein, to said patient in need thereof.

In further addition, the present invention relates to a method for treating, preventing or ameliorating (hyper)proliferative diseases and/or disorders responsive to induction of apoptosis, such as e.g. benign or malignant neoplasia, e.g. cancer, particularly any of those cancer diseases mentioned herein, in a patent comprising administering separately, simultaneously, concurrently, sequentially or chronologically staggered to said patient in need thereof an amount of a first active compound, which is a compound according to the present invention, and an amount of at least one second active compound, said at least one second active compound being a standard therapeutic agent, particularly at least one art-known anti-cancer agent, such as e.g. one or more of those chemotherapeutic and target-specific anti-cancer agents mentioned herein, wherein the amounts of the first active compound and said second active compound result in a therapeutic effect.

In yet further addition, the present invention relates to a method for treating, preventing or ameliorating (hyper)proliferative diseases and/or disorders responsive to induction of apoptosis, such as e.g. benign or malignant neoplasia, e.g. cancer, particularly any of those cancer diseases mentioned herein, in a patient comprising administering a combination according to the present invention.

In addition, the present invention further relates to the use of a composition, combination, formulation, preparation or kit according to this invention in the manufacture of a pharmaceutical product, such as e.g. a commercial package or a medicament, for treating, preventing, or ameliorating (hyper)proliferative diseases, such as e.g. cancer, and/or disorders responsive to the induction of apoptosis, particularly those diseases mentioned herein, such as e.g. malignant or benign neoplasia.

The present invention further relates to a commercial package comprising one or more compounds of the present invention together with instructions for simultaneous, concurrent, sequential or separate use with one or more chemotherapeutic and/or target specific anti-cancer agents, such as e.g. any of those mentioned herein.

The present invention further relates to a commercial package consisting essentially of one or more compounds of the present invention as sole active ingredient together with instructions for simultaneous, concurrent, sequential or separate use with one or more chemotherapeutic and/or target specific anti-cancer agents, such as e.g. any of those mentioned herein.

The present invention further relates to a commercial package comprising one or more chemotherapeutic and/or target specific anti-cancer agents, such as e.g. any of those mentioned herein, together with instructions for simultaneous, concurrent, sequential or separate use with one or more compounds according to the present invention.

The compositions, combinations, preparations, formulations, kits or packages mentioned in the context of the combination therapy according to this invention may also include more than one of the compounds according to this invention and/or more than one of the art-known ant-cancer agents mentioned.

The first and second active ingredient of a combination or kit-of-parts according to this invention may be provided as separate formulations (i.e. independently of one another), which are subsequently brought together for simultaneous, concurrent, sequential, separate or chronologically staggered use in combination therapy; or packaged and presented together as separate components of a combination pack for simultaneous, concurrent, sequential, separate or chronologically staggered use in combination therapy.

The type of pharmaceutical formulation of the first and second active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration.

The amounts of the first and second active ingredients of the combinations, compositions or kits according to this invention may together comprise a therapeutically effective amount for the treatment, prophylaxis or amelioration of a (hyper)proliferative diseases and/or a disorder responsive to the induction of apoptosis, particularly one of those diseases mentioned herein, e.g. benign or malignant neoplasia, especially cancer, like any of those cancer diseases mentioned herein.

In addition, compounds according to the present invention can be used in the pre- or post-surgical treatment of cancer.

In further addition, compounds of the present invention can be used in combination with radiation therapy.

A combination according to this invention can refer to a composition comprising both the compound(s) according to this invention and the other active anti-cancer agent(s) in a fixed combination (fixed unit dosage form), or a medicament pack comprising the two or more active ingredients as discrete separate dosage forms (non-fixed combination). In case of a medicament pack comprising the two or more active ingredients, the active ingredients are preferably packed into blister cards which are suited for improving compliance.

Each blister card preferably contains the medicaments to be taken on one day of treatment. If the medicaments are to be taken at different times of day, the medicaments can be disposed in different sections on the blister card according to the different ranges of times of day at which the medicaments are to be taken (for example morning and evening or morning, midday and evening). The blister cavities for the medicaments to be taken together at a particular time of day are accommodated in the respective range of times of day. The various times of day are, of course, also put on the blister in a clearly visible way. It is also possible, of course, for example to indicate a period in which the medicaments are to be taken, for example stating the times.

The daily sections may represent one line of the blister card, and the times of day are then identified in chronological sequence in this column.

Medicaments which must be taken together at a particular time of day are placed together at the appropriate time on the blister card, preferably a narrow distance apart, allowing them to be pushed out of the blister easily, and having the effect that removal of the dosage form from the blister is not forgotten.

### Biological Investigations

The anti-proliferative/cytotoxic activity of the compounds described herein can be tested on subclones of RKO human colon adenocarcinoma cells (Schmidt et al., Oncogene 19, 2423-2429; 2000) using the Alamar Blue cell viability assay (described in O'Brien et al. Eur J Biochem 267, 5421-5426, 2000). The compounds are dissolved as 10 mM solutions in DMSO and subsequently diluted in semi-logarithmic steps. DMSO dilutions are further diluted 1:100 into Dulbecco's modified Eagle's medium (DMEM) containing 10% fetal calf serum to a final concentration twice as much as the final concentration in the test. RKO subclones are seeded into 96 well flat bottom plates at a density of 4000 cells per well in a volume of 50 μl per well. 24 hours after seeding the 50 μl each of the compound dilutions in DMEM medium are added into each well of the 96 well plate. Each compound dilution is tested as quadruplicates. Wells containing untreated control cells are filled with 50 μl DMEM medium containing 1% DMSO. The cells are then incubated with the substances for 72 hours at 37° C. in a humidified atmosphere containing 5% carbon dioxide. To determine the viability of the cells, 10 μl of an Alamar Blue solution (Biosource) are added and the fluorescence is measured at an extinction of 544 nm and an emission of 590 nm. For the calculation of the cell viability the emission value from untreated cells is set as 100% viability and the emission rates of treated cells are set in relation to the values of untreated cells. Viabilities are expressed as % values.

The corresponding IC₅₀ values of the compounds for anti-proliferative/cytotoxic activity are determined from the concentration-effect curves.

To determine the cell cycle specific mode of action, subclones of RKO colon adenocarcinoma cells (RKOp27 as described by Schmidt et al. in Oncogene 19, 2423-2429; 2000) are seeded into 96 well flat bottom plates at a density of 16000 cells per well in a volume of 50 μl per well in DMEM growth medium with 10% FCS containing 10 μM Ponasterone A. 24 hours after seeding the 50 μl each of the compound dilutions in DMEM medium are added into each well of the 96-well plate. Each compound dilution is tested as quadruplicates. Wells containing untreated control cells are filled with 50 μl DMEM medium containing 1% DMSO. The cells are then incubated with the substances for 72 hours at 37° C. in a humidified atmosphere containing 5% carbon dioxide. To determine the viability of the cells, 10 μl of an Alamar Blue solution (Biosource) are added and the fluorescence is measured at an extinction of 544 nm and an emission of 590 nm. For the calculation of the cell viability the emission value from untreated cells is set as 100% viability and the emission rates of treated cells are set in relation to the values of untreated cells. Viabilities are expressed as % values. Viability is compared of proliferating cells grown in the absence of the inducer Ponasterone A, versus viability of cells arrested by the expression of ectopic p27Kip1 induced by Ponasterone A.

Representative IC₅₀ values for anti-proliferation/cytotoxicity determined in the aforementioned assays follow from the following table A, in which the numbers of the compound correspond to the numbers of the examples.

TABLE A
Anti-proliferative/cytotoxic activity

| | | IC₅₀ RKO p27 |
|---|---|---|
| Compound | IC₅₀ RKO p27 uninduced [μM] | induced [μM] |
| 1 | The IC50 values of these | >100 |
| 2 | listed compounds are all ≦2.5 | >85 |
| 3 | | >100 |
| 4 | | >100 |
| 5 | | >100 |
| 6 | | >100 |
| 7 | | >100 |
| 8 | | >100 |

The induction of apoptosis can be measured by using a Cell death detection ELISA (Roche Biochemicals, Mannheim, Germany). NCI-H460 non-small cell lung cancer cells are seeded into 96 well flat bottom plates at a density of 10000 cells per well in a volume of 50 μl RPMI medium (containing 10% fetal calf serum) per well. 24 hours after seeding the 50 μl each of the compound dilutions in RPMI medium are added into each well of the 96 Well plate. Each compound dilution is tested at least as triplicates. Wells containing untreated control cells are filled with 50 μl RPMI medium containing 1% DMSO. The cells are then incubated with the substances for 24 hours at 37° C. in a humidified atmosphere containing 5% carbon dioxide. As a positive control for the induction of apoptosis, cells are treated with 50 μM Cisplatin (Gry Pharmaceuticals, Kirchzarten, Germany). Medium is then removed and the cells are lysed in 200 μl lysis buffer. After centrifugation as described by the manufacturer, 10 μl of cell lysate is processed as described in the protocol. The degree of apoptosis is calculated as follows: The absorbance at 405 nm obtained with lysates from cells treated with 50 μM cisplatin is set as 100 cpu (cisplatin units), while an absorbance at 405 nm of 0.0 is set as 0.0 cpu. The degree of apoptosis is expressed as cpu in relation to the value of 100 cpu reached with the lysates obtained from cells treated with 50 μM cisplatin.

The effect of the compounds according to the invention may further be assessed by the following tests:

### Microtubule Polymerization Assay

The assay is performed as described in Beckers T., Reissmann T., Schmidt M., Burger A. M., Fiebig H. H. et al. 2-Aroylindoles, a Novel Class of Potent, Orally Active Small Molecule Tubulin Inhibitors, Cancer Research 2002, 62, 3113-3119. Bovine brain tubulin heterodimers (5 μg/μl; 50 μg/assay), provided by Cytoskeleton/TEBU (MAP-rich, order No. ML-113F), are incubated with test compounds in PEM buffer pH 6.6 containing 1 mM GTP in a total volume of 100 μl at 37° C. for 1 h. Concentration dependent inhibition of GTP/heat induced microtubule polymerisation is visualized after staining of polymerized microtubules with 0.1% naphtol blue black solution. The amount of bound dye is determined using a photometer at a wavelength of 600 nm. Inhibition of polymerization is calculated using the GraphPad Prism software. Colchicine or Vincristine are included as positive controls.

### Colchicine Competition Assay

The assay is conducted basically as described in Tahir et al., Biotechniques 29,156f, 2000. Briefly, a 100 μl solution containing 69 μl G-PEM (80 mM Pipes pH6.9, 1 mM MgCl₂, 1 mM EGTA, 5% v/v glycerol), 1 μl compound or DMSO, 10 μl ³H-colchicine (NEN #NET-189; diluted 1:133 with 800 nM unlabeled colchicine dissolved in G-PEM), 10 μl of 10 mM GTP, and 100% biotinylated tubulin (Tebu-bio #T333; dissolved in G-PEM) is incubated for 2 hours at 37° C. 20 μl of yttrium silicate beads (Amersham #RPNQ0012) are then added and the mixture is shaken for 30 minutes at 500 rpm. After settling of the beads for 45 minutes, counts are measured using a Wallac TRILUX 1450 microbeta reader. Values of samples incubated with DMSO instead of compound are set 100%, and the values of competition are calculated as the percentile fraction of the 100% control using graph pad prism software after reduction of background scintillation (sample without biotinylated tubulin, but containing 79 μl G-PEM).

1. A compound of formula I 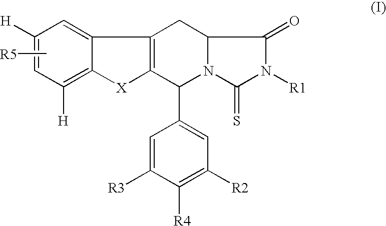
in which
R1 is methyl, ethyl, isopropyl or cyclopropyl,
R2 is methyl, ethyl, halogen, trifluoromethyl, ethoxy or methoxy,
R3 is methyl, ethyl, halogen, trifluoromethyl, ethoxy or methoxy,
R4 is hydrogen,
X is oxygen (O), sulphur (S), or NH,
R5 is hydrogen, methyl, ethyl, halogen, trifluoromethyl, ethoxy or methoxy,
or a salt, stereoisomer or a salt of a stereoisomer thereof.

2. A compound according to claim 1, which is from formula I* 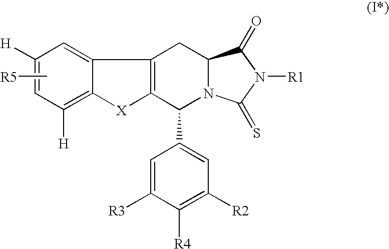
in which
R1 is methyl or ethyl,
R2 is methyl, chlorine, fluorine, trifluoromethyl or methoxy,
R3 is methyl, chlorine, fluorine, trifluoromethyl or methoxy,
R4 is hydrogen,
X is oxygen, sulphur or NH,
R5 is hydrogen, methyl, chlorine, fluorine or trifluoromethyl,
or a salt thereof.

3. A compound according to claim 1, which is from formula I* 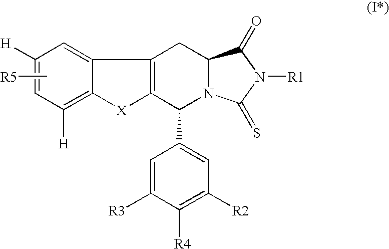
in which
R1 is methyl,
R2 is methyl, chlorine, fluorine, trifluoromethyl or methoxy,
R3 is methyl, chlorine, fluorine, trifluoromethyl or methoxy,
R4 is hydrogen,
X is oxygen, sulphur or NH,
R5 is hydrogen, methyl or fluorine,
or a salt thereof.

4. A compound according to claim 1, which is from formula I* 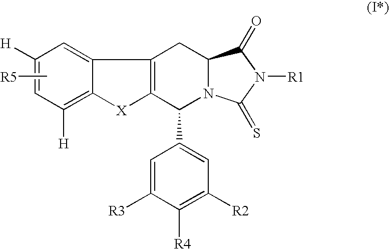
in which
R1 is methyl,
R2 is methyl, chlorine, fluorine or methoxy,
R3 is methyl, chlorine, fluorine or methoxy,
R4 is hydrogen,
X is oxygen, sulphur or NH,
R5 is hydrogen, methyl or fluorine,
or a salt thereof.

5. A compound according to claim 1, which is from formula I* 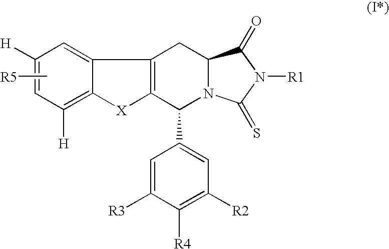
in which
R1 is methyl,
R2 is methyl, chlorine, fluorine or methoxy,
R3 is methyl, chlorine, fluorine or methoxy,
R4 is hydrogen,
X is oxygen, sulphur or NH,
R5 is hydrogen,
or a salt thereof.

6. A compound according to claim 1, which is from formula I* 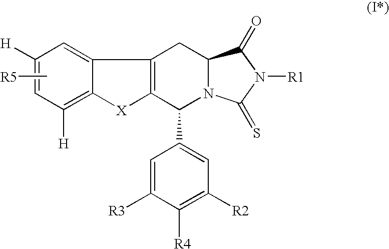
in which
R1 is methyl,
R2 is methyl, chlorine, fluorine or methoxy,
R3 is methyl, chlorine, fluorine or methoxy,
R4 is hydrogen,
X is NH,
R5 is hydrogen,
or a salt thereof.

7. A compound according to claim 1, which is from formula I* 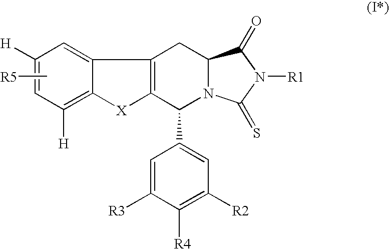
in which
R1 is methyl,
R2 is chlorine, fluorine or methoxy,
R3 is chlorine, fluorine or methoxy,
R4 is hydrogen,
X is oxygen, sulphur or NH,
R5 is hydrogen,
or a salt thereof.

8. A compound according to claim 1, which is from formula I* 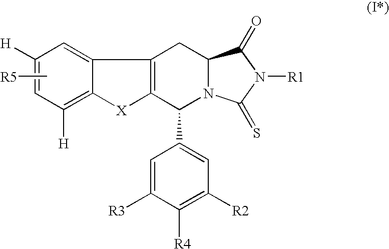
in which
R1 is methyl,
either
R2 is methoxy, and
R3 is methoxy,
or
R2 is chlorine or fluorine, and
R3 is chlorine or fluorine,
R4 is hydrogen,
X is oxygen, sulphur or NH,
R5 is hydrogen,
or a salt thereof.

9. A compound according to claim 1, which is from formula I* 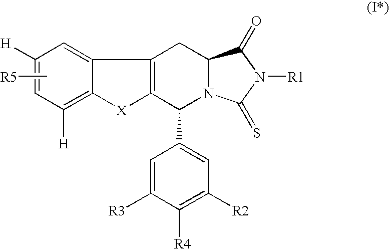
in which
R1 is methyl,
either
R2 is methyl,
R3 is methyl or methoxy,
R4 is hydrogen,
X is NH, and
R5 is hydrogen, methyl, chlorine or fluorine,
or
R2 is methoxy,
R3 is methoxy,
R4 is hydrogen,
X is NH, and
R5 is methyl, chlorine or fluorine,
or a salt thereof.

10. A compound according to claim 1, which is from formula I* 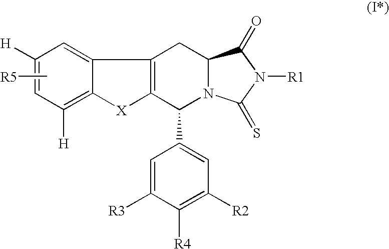
in which
R4 is hydrogen, and
R2, R3 and X have any of the following meanings:

| | X | R2 | R3 |
|---|---|---|---|
| 1.) | NH | Methoxy | Methoxy |
| 2.) | O | Methoxy | Methoxy |
| 3.) | S | methoxy | Methoxy |
| 4.) | NH | chlorine | Chlorine |
| 5.) | O | chlorine | Chlorine |
| 6.) | S | chlorine | Chlorine |
| 7.) | NH | fluorine | Fluorine |
| 8.) | O | fluorine | fluorine |
| 9.) | S | fluorine | fluorine |
| 10.) | NH | chlorine | methoxy |
| 11.) | O | chlorine | methoxy |
| 12.) | S | chlorine | methoxy |
| 13.) | NH | fluorine | methoxy |
| 14.) | O | fluorine | methoxy |
| 15.) | S | fluorine | methoxy |
| 16.) | NH | chlorine | fluorine |
| 17.) | O | chlorine | fluorine |
| 18.) | S | chlorine | fluorine |
| 19.) | NH | methyl | methyl |
| 20.) | O | methyl | methyl |
| 21.) | S | methyl | methyl |
| 22.) | NH | methyl | methoxy |
| 23.) | O | methyl | methoxy |
| 24.) | S | methyl | methoxy |
| 25.) | NH | methyl | chlorine |
| 26.) | O | methyl | chlorine |
| 27.) | S | methyl | chlorine |
| 28.) | NH | methyl | fluorine |
| 29.) | O | methyl | fluorine |
| 30.) | S | methyl | fluorine |

or a salt thereof.

11. A compound of formula I according to claim 1, which is selected from the group consisting of
(3aS,10R)-10-(3,5-Dimethoxy-phenyl)-2-methyl-1-thioxo-1,2,3a,4,9,10-hexahydro-2,9,10a-triaza-cyclopenta[b]fluoren-3-one,
(3aS,10R)-10-(3,5-Dimethoxy-phenyl)-2-methyl-1-thioxo-1,2,4,10-tetrahydro-3aH-9-thia-2,10a-diaza-cyclopenta[b]fluoren-3-one,
(3aS,10R)-10-(3,5-Dichloro-phenyl)-2-methyl-1-thioxo-1,2,3a,4,9,10-hexahydro-2,9,10a-triaza-cyclopenta[b]fluoren-3-one,
(3aS,10R)-10-(3,5-Difluoro-phenyl)-2-methyl-1-thioxo-1,2,3a,4,9,10-hexahydro-2,9,10a-triaza-cyclopenta[b]fluoren-3-one,
(3aS,10R)-10-(3,5-Dimethyl-phenyl)-2-methyl-1-thioxo-1,2,3a,4,9,10-hexahydro-2,9,10a-triaza-cyclopenta[b]fluoren-3-one,
(3aS,10R)-10-(3,5-Dimethoxy-phenyl)-7-fluoro-2-methyl-1-thioxo-1,2,3a,4,9,10-hexahydro-2,9,10a-triaza-cyclopenta[b]fluoren-3-one,
(3aS,10R)-10-(3,5-Dimethoxy-phenyl)-2,5-dimethyl-1-thioxo-1,2,3a,4,9,10-hexahydro-2,9,10a-triaza-cyclopenta[b]fluoren-3-one
(3aS,10R)-10-(3,5-Dimethoxy-phenyl)-2,7-dimethyl-1-thioxo-1,2,3a,4,9,10-hexahydro-2,9,10a-triaza-cyclopenta[b]fluoren-3-one,
and salts thereof.

12. (canceled)

13. A pharmaceutical composition comprising one or more compounds according to claim 1, or a pharmaceutically acceptable salt, stereoisomer or salt of a stereoisomer thereof, together with a pharmaceutically acceptable auxiliary and/or excipient.

14. (canceled)

15. A method for treating, preventing or ameliorating (hyper)proliferative diseases and/or disorders responsive to induction of apoptosis, in a mammal comprising administering a therapeutically effective and tolerable amount of one or more compounds according to claim 1, or a pharmaceutically acceptable salt, stereoisomer or salt of a stereoisomer thereof to said mammal in need thereof.

16. A combination comprising
a first active ingredient, which is at least one compound according to claim 1, or a pharmaceutically acceptable salt, stereoisomer or salt of a stereoisomer thereof, and
a second active ingredient, which is at least one anti-cancer agent selected from the group consisting of chemotherapeutic anti-cancer agents and target-specific anti-cancer agents,
for separate, sequential, simultaneous, concurrent or chronologically staggered use in therapy.

17. A method for treating, preventing or ameliorating hyperproliferative diseases and/or disorders responsive to induction of apoptosis in a patient comprising administering separately, simultaneously, concurrently, sequentially or chronologically staggered to said patient in need thereof
an amount of a first active compound, which is a compound according to claim 1, or a pharmaceutically acceptable salt, stereoisomer or salt of a stereoisomer thereof, and
an amount of at least one second active compound, said second active compound being an anti-cancer agent selected from the group consisting of chemotherapeutic anti-cancer agents and target-specific anti-cancer agents,
wherein the amounts of the first active compound and said second active compound result in a therapeutic effect.

18. The combination according to claim 16, in which said chemotherapeutic anti-cancer agents are selected from the group consisting of (i) alkylating/carbamylating agents; (ii) platinum derivatives; (iii) antimitotic agents/tubulin inhibitors; (iv) topoisomerase inhibitors; (v) pyrimidine antagonists; (vi) purin antagonists; and (vii) folic acid antagonists.

19. The combination according to claim 16, in which said target-specific anti-cancer agents are selected from (i) kinase inhibitors; (ii) proteasome inhibitors; (iii) histone deacetylase inhibitors; (iv) heat shock protein 90 inhibitors; (v) vascular targeting agents (VAT); (vi) monoclonal antibodies; (vii) oligonucleotide based therapeutics; (viii) Toll-like receptor/TLR 9 agonists; (ix) protease inhibitors; (x) hormonal therapeutics; (xi) bleomycin; (xii) retinoids; (xiii) DNA methyltransferase inhibitors; (xiv) alanosine; (xv) cytokines; interferons; and death receptor agonists.

20. The combination according to claim 16, in which said cancer is selected from the group consisting of cancer of the breast, bladder, bone, brain, central and peripheral nervous system, colon, endocrine glands, esophagus, endometrium, germ cells, head and neck, kidney, liver, lung, larynx and hypopharynx, mesothelioma, sarcoma, ovary, pancreas, prostate, rectum, renal, small intestine, soft tissue, testis, stomach, skin, ureter, vagina and vulva; inherited cancers, retinoblastoma and Wilms tumor; leukemia, lymphoma, non-Hodgkins disease, chronic and acute myeloid leukaemia, acute lymphoblastic leukemia, Hodgkins disease, multiple myeloma and T-cell lymphoma; myelodysplastic syndrome, plasma cell neoplasia, paraneoplastic syndromes, cancers of unknown primary site and AIDS related malignancies.
